(19) United States
(12) Patent Application Publication
(10) Pub. No.: US 2022/0079761 A1
(43) Pub. Date: Mar. 17, 2022

(54) Atrioventricular Valve Repair Ring

(71) Applicant: The Medical College of Wisconsin, Inc., Milwaukee, WI (US)

(72) Inventor: Paul J. Pearson, Mequon, WI (US)

(21) Appl. No.: 17/473,559

(22) Filed: Sep. 13, 2021

Related U.S. Application Data
(60) Provisional application No. 63/077,180, filed on Sep. 11, 2020.

(51) Int. Cl. A61F 2/24

(52) U.S. Cl. CPC A61F 2/2463; A61F 2/2445; A61F 2250/0098; A61F 2230/0008; A61F 2210/0014

(57) ABSTRACT

An atrioventricular valve repair ring include an annular or otherwise arcuate shaped ring having a core that is partially or fully enclosed in a sheath. A barrier spans the inner volume of the ring. The barrier can be composed of interwoven filaments, a mesh screen, a porous sheet, and so on. When deployed in a patient's heart, the atrioventricular valve repair ring circumscribes the annulus of the atrioventricular valve. Blood is permitted to flow through the barrier while containing any prolapsing leaflet segments within the appropriate coaptation plane.

### CROSS-REFERENCE TO RELATED APPLICATIONS

This application claims priority to U.S. Patent Application No. 63/077,180 filed Sep. 11, 2020, and entitled, “Atrioventricular Valve Repair Ring,” which is hereby incorporated by reference in its entirety.

### STATEMENT REGARDING FEDERALLY SPONSORED RESEARCH

N/A

### BACKGROUND

Mitral valve regurgitation is frequently caused by myxomatous disease with prolapse of the leaflets, leading to annular dilatation. While mitral repair can result in excellent outcomes with a low mortality risk and long-term durability, surgical techniques are currently difficult to reproduce, particularly in centers where surgeons do not have access to a high volume of procedures.

### SUMMARY OF THE DISCLOSURE

The present disclosure addresses the aforementioned drawbacks by providing an atrioventricular valve repair ring that includes a ring and a barrier spanning the inner volume of the ring. The ring includes a core and a sheath. The core has an arcuate shape that circumscribes the inner volume of the ring. The sheath surrounds at least a portion of the core. The barrier is coupled to the sheath and spans the inner volume of the ring, where the barrier is constructed to permit blood to flow therethrough while preventing atrioventricular valve leaflets from passing beyond the barrier.

In some embodiments, a core can have an annular shape and can define a continuous periphery of a ring.

In some embodiments, a core can have an elliptical annular shape.

In some embodiments, a core can define a discontinuous periphery of a ring.

In some embodiments, a core can be composed of a shape-memory material.

In some embodiments, a core can be composed of a shape-memory alloy.

In some embodiments, a core can be composed of nitinol.

In some embodiments, a core can be composed of a shape-memory polymer.

In some embodiments, a sheath can be composed of a textile material.

In some embodiments, a textile material can be a polymer-based textile material.

In some embodiments, a polymer-based textile material can include expanded polytetrafluoroethylene (PTFE).

In some embodiments, a sheath can fully enclose a core.

In some embodiments, a barrier can include a plurality of filaments.

In some embodiments, a plurality of filaments can be interwoven to form a mesh.

In some embodiments, at least some of a plurality of filaments can be looped around others of the plurality of filaments when forming a mesh.

In some embodiments, a plurality of filaments can be interwoven using a symmetrical weave pattern.

In some embodiments, a plurality of filaments can be interwoven using an asymmetrical weave pattern.

In some embodiments, a plurality of filaments can be asymmetrically distributed within an inner volume of a ring such that some portions of a barrier have a higher density of filaments than other portions of the barrier.

In some embodiments, a plurality of filaments can be coupled to a sheath by stitching the plurality of filaments to the sheath.

In some embodiments, a plurality of filaments can be composed of expanded polytetrafluoroethylene (PTFE).

In some embodiments, a barrier can include a mesh screen.

In some embodiments, a mesh screen can include a regularly spaced grid of holes in a screen material.

In some embodiments, a screen material can be composed of a biocompatible polymer.

In some embodiments, a mesh screen can include asymmetrically distributed holes in a screen material, such that some portions of the mesh screen have a higher density of holes than other portions of the mesh screen.

In some embodiments, asymmetrically distributed holes can be distributed in a pattern that accounts for patient-specific anatomy.

In some embodiments, a barrier can include a porous sheet.

In some embodiments, a atrioventricular valve repair ring can include at least one marker coupled to the ring in order to facilitate placement of the ring during a surgical procedure.

In some embodiments, at least one marker can be composed of a radiopaque material.

In some embodiments, at least one marker can include a plurality of markers that are distributed about an outer surface of a sheath.

Some embodiments of the disclosure provide an atrioventricular valve repair device. The device can include a frame, and a barrier coupled to the frame. The frame can surround at least a portion of the frame. The barrier can have one or more holes each of which can be configured to receive blood therethrough. The device can be configured to allow passage of blood from an atrium through the one or more holes of the barrier, through an atrioventricular valve, and into a ventricle during contraction of the atrium. The barrier can be configured to block advancement of one or more leaflets of the atrioventricular valve from extending farther into the atrium away from the ventricle during contraction of the ventricle.

In some embodiments, a frame can have a first end and a second end opposite the first end. A barrier can have a first end and an opposite second end. The first end of the barrier can be coupled to the first end of the frame. The second end of the barrier can be coupled to the second end of the frame.

In some embodiments, a frame can be configured to engage at least a portion of an atrium wall of an atrium.

In some embodiments, a portion of the frame can be configured to contour a curvature of an atrium wall of an atrium.

In some embodiments, a frame can include a core, and a sheath at least partially surrounding the core. The sheath can be formed from a different material than the core.

In some embodiments, a barrier can be coupled to at least one of a core, or a sheath.

In some embodiments, a barrier can include a plurality of filaments with each filament overlapping an adjacent filament according to a pattern. Adjacent filaments can collectively define a hole of the barrier.

In some embodiments, each filament can have a higher tensile strength than a tensile strength of a frame.

In some embodiments, each filament can be tensilely loaded. The tensile loading of each filament can be greater than a tensile loading of the frame.

In some embodiments, a frame is not tensilely loaded.

In some embodiments, each filament can have a cross-section that is smaller than a cross-section of the frame.

In some embodiments, each filament can have a uniform cross-section along its length. A cross-section of a frame can be uniform along the entire peripheral extent of the frame.

In some embodiments, one end of each filament can be coupled to one end of a frame. An opposing end of each filament can be coupled to an opposing end of the frame.

In some embodiments, a frame can be configured to be compressed from a first configuration to a second configuration. The frame can be configured to expand from the second configuration to the first configuration to engage an atrium wall of an atrium.

In some embodiments, an atrioventricular valve repair device can be configured to be positioned within an atrium on a first side of an atrioventricular valve that is farther away from a ventricle than a second side of the atrioventricular valve opposite the first side of the atrioventricular valve. The atrioventricular valve repair device can be configured to be coupled to a wall of the heart.

Some embodiments of the disclosure provide a method of repairing an atrioventricular valve of a patient. The method can include placing an atrioventricular valve repair device into the patient, advancing the device until the device is positioned within the atrium of the patient on a first side of the atrioventricular valve that is farther from the ventricle of the patient than a second side of the atrioventricular valve, engaging the device with a heart of the patient, coupling the device to the heart of the patient, passing blood through the device during contraction of the atrium, and mitigating backflow of blood from the ventricle and into the atrium during contraction of the ventricle.

In some embodiments, the method can include blocking advancement of one or more leaflets of an atrioventricular valve from extending farther into an atrium away from a ventricle during contraction of the ventricle to mitigate backflow of blood.

In some embodiments, the method can include contacting a barrier of an atrioventricular valve repair device with one or more leaflets of the atrioventricular valve to block further extension of the one or more leaflets.

In some embodiments, the method can include passing blood from an atrium through one or more holes of a barrier of an atrioventricular valve repair device, through an atrioventricular valve, and into a ventricle during contraction of the atrium. The one or more holes can be smaller than a leaflet of the atrioventricular valve.

In some embodiments, backflow can be mitigated without modifying an atrioventricular valve.

In some embodiments, backflow can be mitigated without radially compressing one or more leaflets of an atrioventricular valve in a direction towards a wall of a heart.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIG. 1 is a plan view of an example atrioventricular valve repair ring having a continuous periphery and a barrier composed of a plurality of filaments, according to some embodiments described in the present disclosure.

FIG. 2 is a plan view of an example atrioventricular valve repair ring having a discontinuous periphery and a barrier composed of a plurality of filaments, according to some embodiments described in the present disclosure.

FIG. 3 shows an example filament spanning the inner volume of a atrioventricular valve repair ring, where the filament is coupled to a sheath surrounding a core of the atrioventricular valve repair ring, such as by stitching the filament into the sheath.

FIG. 4 shows an axial cross-sectional view of a heart.

FIG. 5 shows a top view of the device of FIG. 1 with the frame in a first configuration, and with the frame in a second configuration.

FIG. 6 shows a top view of another atrioventricular valve repair device.

FIG. 7 shows an example of looping one filament around another when constructing a barrier according to some embodiments described in the present disclosure.

FIG. 8 shows an example of another atrioventricular valve repair device in a first configuration, and in a second configuration.

FIG. 9 is a plan view of an example atrioventricular valve repair ring having a continuous periphery and a barrier composed of a mesh scree, according to some embodiments described in the present disclosure.

FIG. 10 is a plan view of an example atrioventricular valve repair ring having a continuous periphery and a barrier composed of a porous sheet, according to some embodiments described in the present disclosure.

FIG. 11 shows an example of an atrioventricular valve repair ring according to some embodiments described in the present disclosure, which has been deployed in a patient's heart.

FIG. 12 shows a flowchart of a process for repairing an atrioventricular valve.

### DETAILED DESCRIPTION

Described here are atrioventricular valve repair rings, which when deployed in a patient's heart can mitigate (or prevent) atrioventricular valve (e.g., mitral valve or tricuspid valve) leaflets from prolapsing into the atria (e.g., left atrium or right atrium). The atrioventricular valve repair rings described in the present disclosure have the advantage of increasing the simplicity of atrioventricular valve repair, while at the same time preserving the durable benefits of surgical repair.

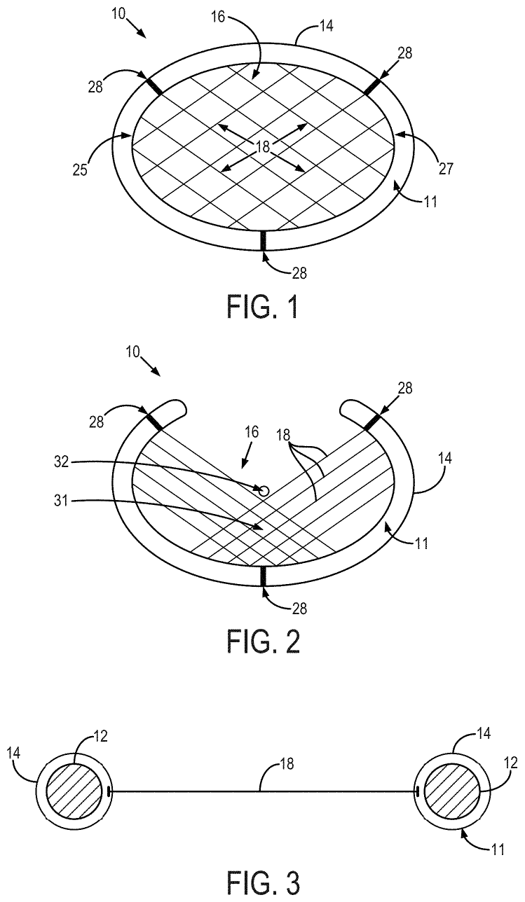

As shown in FIG. 1, an atrioventricular valve repair ring (or device) according to various embodiments described in the present disclosure generally includes a ring (or similarly ring-like structure) circumscribing an inner volume. A barrier spans the inner volume, such that when the ring is deployed into a patient's heart, such as around the annulus of the atrioventricular valve on the inflow (i.e., atrial) side of the atrioventricular valve, the barrier contains any prolapsing leaflet segments within the appropriate coaptation plane while still permitting blood to flow through the barrier. In this way, the barrier eliminates atrioventricular regurgitation without the requirement for leaflet resection, chordal transfer/neo chords, or other complex surgical repair techniques. Further still, the ring can be structured similarly to an annuloplasty band, thereby stabilizing the annulus and retaining the durability of a standard atrioventricular valve repair.

FIG. 1 shows a plan view of an atrioventricular valve repair device 10 (which will also be referred to herein in a specific implementation as a ring 10) according to some embodiments described in the present disclosure. The device 10 can include a frame 11 that can include a core 12 having a substantially (i.e., deviating by less than 10 percent) annular shape, and a sheath 14 that covers the core 12. The device 10 can also include a barrier 16 spanning an internal volume of the atrioventricular valve repair ring 10, which reduces (e.g., prevents) atrioventricular valve leaflets from prolapsing into the atrium while still permitting blood to flow therethrough. As will be described, the barrier 16 (and other barriers described herein) can have many different forms, including but not limited to a plurality of filaments, a mesh screen, an additively manufactured mesh (e.g., a 3D printed mesh), or the like. Thus, the barrier 16 is generally a porous or otherwise permeable barrier 16 that enables blood flow through pores, apertures, or other openings in the barrier 16, while mitigating (e.g., prohibiting) movement of the atrioventricular valve leaflets beyond the structure of the barrier 16. For instance, the barrier 16 may have openings formed therein, as in the case of the barrier 16 being constructed as a webbing or net of intertwined filaments, where the openings are the spaces between the filaments. As another example, the barrier 16 may have apertures formed therein, as in the case of the barrier 16 being formed as a mesh screen or 3D printed structure having apertures formed therein. As still another example, the barrier 16 may have pores formed therein, as in the case of the barrier 16 being formed as a porous sheet. The core 12, sheath 14, and barrier 16 can be composed of biocompatible materials, such as those example materials described below.

In some embodiments, the barrier 16 can define one or more holes (e.g., one, two, three, four, etc.) directed through the barrier 16 that provide fluid communication between an upper end of the device 10 and a lower end of the device 10. For example, each hole can be configured to receive blood therethrough (e.g., in direction from the upper end to the lower end of the device 10), and each hole can be smaller than a leaflet of an atrioventricular valve (e.g., smaller than a free end of the atrioventricular valve). In this way, each hole can allow blood to flow through according to the natural blood flow path through the heart (e.g., from the atrium, through the barrier 16 of the device 10, and into the ventricle), while reducing regurgitation of blood (e.g., backflow of blood) from the ventricle through the atrioventricular valve and into the atrium (e.g., by one or more leaflets of the atrioventricular valve contacting the barrier 16 and preventing further advancement of the one or more leaflets past the barrier 16 into the atrium thereby mitigating prolapsing of the valve leaflets). In some cases, each hole of the barrier 16 can be configured to prevent passage of a free end of leaflet through the respective hole. In some configurations, one or more holes of the barrier 16 can receive a portion of a leaflet of the atrioventricular valve (e.g., when the ventricle is contracting), but with each leaflet being prevented from prolapsing.

In general, the core 12 can have an arcuate shape, which in some instances may define a substantially annular shape for the atrioventricular valve repair ring 10. As one example, the core 12 can be a circular annular ring. As another example, the core 12 can be an elliptical annular ring. In still other examples, the core 12 can have other annular shapes. The core 12 may define a continuous periphery for the atrioventricular valve repair ring 10 as shown in FIG. 1, or a discontinuous periphery for the atrioventricular valve repair ring 10, as shown in FIG. 2.

The core 12, and thus the atrioventricular valve repair ring 10, is generally arranged within a plane that is perpendicular to a central axis of the atrioventricular valve repair ring 10. For instance, the central axis generally extends through the centroid of the atrioventricular valve repair ring 10 when viewed in a plan view. In this arrangement, the average inflow-outflow direction of blood flowing through the atrioventricular valve is generally parallel with the central axis. Although the atrioventricular valve repair rings 10 described in the present disclosure are generally three-dimensional structures, portions of the atrioventricular valve repair rings 10 lie in a plane perpendicular to this blood flow axis.

In some embodiments, the core 12 is made from a flexible material, such as a flexible elastic material. As one non-limiting example, the core 12 can be composed of a shape-memory alloy, such as nitinol. In other examples, the core 12 can be composed of a polymer, which in some instances may be a shape-memory polymer. In these ways, the core 12 can have a first shape prior to being implanted into a patient (e.g., with the core 12 being at a first temperature that is below body temperature such as below substantially 37° C.), and can spontaneously move into a second shape after being implanted into a subject (e.g., with the core 12 being at a second temperature higher than the first temperature). In some cases, the second shape can at least partially conform to the peripheral shape of an atrium of the patient (e.g., relative to an axial cross-section of the atrium of the patient). For example, the entire (or a portion of the) periphery of the core 12 can contour the curvature of a section (or the entire) of the wall of the periphery of the atrium (with the atrium in an axial cross-section).

In some other embodiments, the core 12 can alternatively be made from a rigid or semi-rigid material. For example, the core 12 being rigid can be defined as the shape of the core 12 deviating by less than ten percent from an unloaded state (e.g., when placed in the ambient environment). As a more specific example, with the core 12 being rigid, the inner volume defined by the boundary of the core 12 in the unloaded state can deviate by less than ten percent when loaded (e.g., after the device is implanted in the subject). In other configurations, the core 12 being semi-rigid (or in other words semi-flexible) can include the shape of the core 12 deviating by greater than ten percent from the unloaded state. In some cases, this can include the inner volume defined by the boundary of the core 12 deviating greater than ten percent when loaded. In some configurations, the core 12 can be compressed prior to being engaged with the wall of the atrium, and can partially (or fully) retract to press the frame 11 against the wall of the atrium thereby forcing the device 10 in place (e.g., in a spring-like manner). In some configurations, while the core 12 has been described as being rigid, semi-rigid, flexible, etc., the frame 11, which can include the core 12, can also be rigid, semi-rigid, flexible, etc., which can be defined in a similar manner as the core 12.

In some configurations, the periphery of the frame 11 of the device 10 can conform (e.g., when the frame 11 moves from a loaded state to an unloaded state) to a portion (or the entire) of a periphery (e.g., in an axial view) of the wall of an atrium of the patient. In this way, the frame 11 can retract against the wall of the atrium to further secure the device 10 in place. In other cases, the frame 11, in an unloaded state (e.g., prior to being implanted in the patient), can have a peripheral shape that corresponds to the shape of the wall of the atrium. In this way, the frame 11 does not have to retract, but rather abuts against the wall of the atrium thereby securing the device 10 to the atrium without imparting a force against the wall of the atrium (e.g., for more delicate heart tissue).

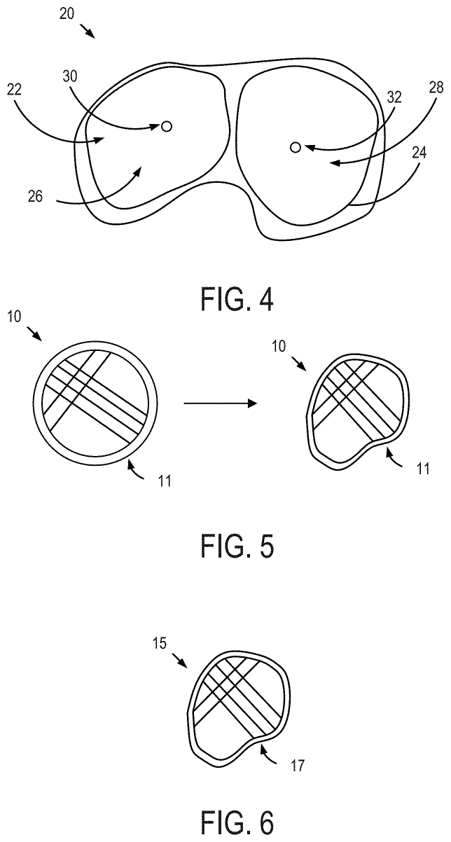

FIG. 4 shows an axial cross-sectional view of a heart 20. As shown in FIG. 4, the heart 20 includes a right atrium 22, a left atrium 24, a tricuspid valve 26 (e.g., that has one or more prolapsed leaflets), and a mitral valve 28 (e.g., that has one or more prolapsed leaflets). A first axial axis 30 is perpendicular to the horizontal (or axial) plane and extends from the right atrium into the right ventricle. Similarly, a second axial axis 32 is perpendicular to the horizontal plane and extends from the left atrium into the left ventricle.

FIG. 5 shows a top view of the device 10 with the frame 11 in a first configuration, for example, prior to being implanted in the patient. In the first configuration, the frame 11 can have a first peripheral shape, which as illustrated in FIG. 5 can be an arcuate shape (e.g., a circle), however the frame 11 in the first configuration could have other shapes (e.g., a square, a rectangle, an oval, an ellipse, etc.). The device 10 can then move from the first configuration (left portion of FIG. 5) into a second configuration (right portion of FIG. 5) different from the first configuration. In the second configuration, the frame 11 has a second peripheral shape that is different than the first peripheral shape. In particular, the second peripheral shape can partially (or entirely) correspond to the peripheral shape of a wall of an atrium within the axial plane of the atrium (e.g., the axial plane of FIG. 4). For example, the peripheral shape of the device 10 can partially (or entirely) correspond to the peripheral shape of the wall of the right atrium 22 that is defined around the axial axis 30 and within the axial plane of the right atrium 22 (e.g., with the axial plane being proximal to the bicuspid valve 26). As another example, the peripheral shape of the device 10 can partially (or entirely) correspond to the peripheral shape of the wall of the left atrium 24 that is defined around the axial axis 32 and within the axial plane of the left atrium 34 (e.g., with the axial plane being proximal to the mitral valve 28). In some configurations, the frame 11 of the device 10 can spontaneously move from the first configuration to the second configuration (e.g., when adjusting the temperature of the device 10), or can be loaded to move from the first configuration to the second configuration (e.g., by compressing the frame 11). Regardless of the configuration, by moving from the first configuration to the second configuration, the frame 11, when placed into an atrium of the patient, can retract against the atrium wall, which can better secure the device 10 to the atrium wall, and can accommodate for different atrium anatomies (e.g., across different patients).

FIG. 6 shows a top view of an atrioventricular valve repair device 15, which can be implemented in a similar manner as the atrioventricular valve repair device 10 described herein. Thus, the atrioventricular valve repair device 10 also pertains to the atrioventricular valve repair device 15. As shown in FIG. 6, the device 15 can include a frame 17, which can have a peripheral shape that partially (or entirely) corresponds to the peripheral shape of an atrium wall (e.g., defined around an axial axis of an atrium that includes the atrium wall and within an axial plane of the atrium). In some cases, the frame 17 of the device 15 does not move from a first configuration having a first peripheral shape to a second configuration having a second peripheral shape. Rather, the peripheral shape of the frame 17 is substantially maintained, irrespective of loading of the frame 17. For example, the peripheral shape of the frame 17 can be substantially the same prior to implanting the device in the patient and after implanting the device 15 in the patient. In other cases, the frame 17 having a peripheral shape that substantially mimics the curvature of the atrium of a patient, can move from a first configuration (e.g., the one as illustrated in FIG. 6), to a second configuration that has a different peripheral shape. For example, the frame 17 can accommodate for deviations in curvature between the frame 17 and the atrial wall, so that the curvature of the peripheral shape of the frame 17 in the second configuration (e.g., after being implanted or engaged with the atrium of the patient) is substantially the same as the curvature of the atrium wall of the patient. In some configurations, the frame 17 can be rigid, semi-rigid, or flexible.

In some embodiments, the cross-sectional area defined by the frame 17 (e.g., the area bounded by the frame 17) prior to engaging the atrium wall can be larger than the axial cross-sectional area of the atrium in which the device 15 is engaged with (e.g., implanted into). In other words, the axial cross-section of the atrium that is in contact with the device 15 can be smaller than the cross-sectional area defined by the frame 17 prior to the frame 17 engaging with the atrium. In this way, the atrial wall can retract around the frame 17 thereby stabilizing the device 15 and preventing dislodgement of the device 15.

Referring back to FIGS. 1-3, the sheath 14 is arranged about the core 12, such that the core 12 is fully or partially enclosed within the sheath 14. The sheath 14 can be composed of polymer-based textile materials or other such textile materials that are knit and/or woven to form the sheath 14. As one non-limiting example, the sheath 14 can be composed of an expanded polytetrafluoroethylene (“PTFE”) material, such as GORE-TEX® (W. L. Gore & Associates, Inc.; Newark, Del., United States). In other example, the sheath 14 may be composed of polyethylene terephthalate (“PETE”) materials, such as DACRON® (Invista; Wichita, Kans., United States). In other embodiments, the sheath 14 can be composed of other expandable materials, such as elastomeric membranes.

In some embodiments, the frame 11 can have a uniform cross-section along the entire peripheral extent of the frame 11 (e.g., along a peripheral length of the frame 11). Thus, the core 12 and the sheath 14 can each have a uniform cross-section along the entire peripheral extent of the frame 11. In other configurations, the frame 11 can have a non-uniform cross-section along the entire peripheral extent of the frame 11 (e.g., the cross-section can increase from one end to the other, and vice versa). Thus, the core 12 can have a non-uniform cross-section along the entire peripheral extent of the frame 11, and the sheath 14 can have a non-uniform cross-section along the entire peripheral extent of the frame 11, which can span differently than the how the cross-section varies along the core 12.

As noted, the barrier 16 spans an inner volume of the atrioventricular valve repair ring 10, such as the inner volume defined by the inner periphery of the frame 11 (e.g., the core 12 and sheath 14). For example, the barrier 16 can be coupled to opposing ends of the frame 11 (e.g., at the sheath 14, or the core 12), such as opposing inner ends 25, 27 of the frame 11 (e.g., that are defined at opposing sides of an inner surface of the frame 11). In some embodiments, such as those shown in FIGS. 1 and 2, the barrier 16 is constructed from a plurality of filaments 18 that are coupled to the sheath 14 surrounding the core 12 of the atrioventricular valve repair ring 10. For instance, the filaments 18 can be woven into the inner periphery of the sheath 14, as shown in FIG. 3. In other configurations, the barrier 16 can be coupled to the core 12. For example, a portion of the barrier 16 can be wrapped around the core 12 at, for example, opposing ends of the core 12. As a more specific example, each filament 18 can be wrapped around the core 12 at, for example, opposing ends of the core 12. In this way, the sheath 14 can enclose at least a portion of the core 12 and at least a portion of the barrier 16, and because the barrier 16 is anchored to the core 12 (e.g., which can have stronger material properties as the sheath 14), the barrier 16 can be less likely to detach from the frame 11 (e.g., via detaching from the sheath 14).

In some embodiments, each filament 18 can have a substantially uniform cross-section along its length, and each filament 18 can have a cross-section that is smaller than a cross-section of the frame 11. For example, each filament 18 can have a cross-section that is smaller than a cross-section of a core 12, or a cross-section of the sheath 14. In some configurations, each filament 18 when coupled to the frame 11 (e.g., at opposing sides of the frame 11) can be tensilely loaded (e.g., by stretching the respective filament 18), which can block further advancement of the one or more leaflets of the atrioventricular valve.

As one example, the filaments 18 can be composed of PTFE. For example, the barrier 16 can be composed of 6-0 PTFE filaments 18 that are interwoven to generally form a mesh that enables blood to flow therethrough, while containing atrioventricular valve leaflet segments within the appropriate coaptation plane, thereby eliminating atrioventricular regurgitation.

The filaments 18 can be interwoven with each other in order to improve the structural stability of the barrier 16. In some configurations, the filaments 18 can be interwoven using an over-under pattern, which may include a symmetrical or asymmetrical over-under pattern. As an example, a symmetrical over-under pattern may include an “over one, under one” pattern, an “over two, under two” pattern, and so on. Similarly, an asymmetrical over-under pattern may include an “over one, under two” pattern, an “over two, under one” pattern, and so on.

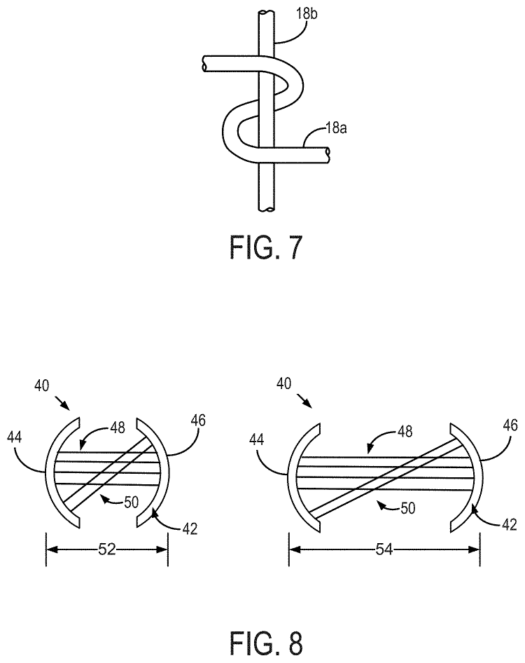

In some other configurations, the filaments 18 can be interwoven such that when filaments spanning one direction (e.g., filament 18a in FIG. 7) pass filaments spanning a perpendicular or otherwise non-parallel direction (e.g. filament 18b in FIG. 7), then one of those filaments 18a is looped and/or wrapped around the other filament 18b, as shown in FIG. 7. This configuration can provide additional stability in the barrier 16.

To facilitate placement of the atrioventricular valve repair ring 10 during a surgical procedure, one or more markers 28 (e.g., one, two, three, four, etc., markers) can be coupled to or otherwise arranged on the atrioventricular valve repair ring 10. As one non-limiting example, as shown in FIG. 1, three such markers 28 are coupled to or otherwise arranged on the atrioventricular valve repair ring 10. The markers 28 can be, for instance, radiopaque markers that are coupled to or otherwise arranged on the frame 11 (e.g., on the sheath 14 that surrounds the core 12 of the atrioventricular valve repair ring 10). The markers 28 can be arranged symmetrically or asymmetrically around the periphery of the atrioventricular valve repair ring 10. In the example shown in FIG. 1, the markers 28 are spaced apart from each other by 120 degrees relative to a central axis of the atrioventricular valve repair ring 10. It will be appreciated that the atrioventricular valve repair ring 10 can similarly be provided with fewer than three markers 28, or more than three markers 28.

Referring again to FIG. 1, in one example configuration, the atrioventricular valve repair ring 10 can include a core 12 that defines a continuous periphery. In this example, the barrier 16 is constructed from a plurality of filaments 18 that each span the inner volume of the core 12. Each filament 18 is coupled to the sheath 14 that surrounds the core 12. In some configurations, the barrier 16 (and each filament 18) can be positioned between opposing axial ends of the frame 11 (e.g., the axial ends being defined relative to an axial axis that is parallel to the central axis of the device 10). In other configurations, the barrier 16 can be coupled to the frame 11 at one axial end of the frame 11 so that the barrier 16 is positioned above a first axial end of the frame 11, and above (or at) a second axial end of the frame 11 that is opposite the first axial end of the frame 11. In some cases, the barrier 16 can be more structurally sound when the barrier 16 is coupled to the frame 11 and is positioned between opposing axial ends of the frame 11.

The filaments 18 can be equally spaced, or alternatively can be asymmetrically arranged within the inner volume of the atrioventricular valve repair ring 10. For instance, the filaments 18 can be arranged such that a higher density of filaments 18 is included in areas where greater support is desired. Each filament 18 can span the inner volume of the atrioventricular valve repair ring 10 by originating on one side of the atrioventricular valve repair ring 10 and extending to a point on the opposing side of the atrioventricular valve repair ring 10. In some examples, the filaments 18 can span the inner volume of the atrioventricular valve repair ring 10 as geometrical chords of a circular or elliptical cross-section defined by the atrioventricular valve repair ring 10.

Referring to FIG. 2, in another example configuration, the atrioventricular valve repair ring 10 can include a frame 11 (e.g., and core 12) that defines a discontinuous periphery. In this example, the barrier 16 is constructed from a plurality of filaments 18 that each span the inner volume of the core 12. Each filament 18 is coupled to the sheath 14 that surrounds the core 12.

As described above, the filaments 18 can be equally spaced, or alternatively can be asymmetrically arranged within the inner volume of the atrioventricular valve repair ring 10. For instance, the filaments 18 can be arranged such that a higher density of filaments 18 is included in areas where greater support is desired. In particular, as shown in FIG. 3, the barrier 16 can include a central region 31 that is located proximal to the centroid 32 of the frame 11, which has a higher density of filaments 18 (e.g., or less holes defined by the barrier 16) than other portions of the device 10 (e.g., regions of the barrier 16 positioned closer to the frame 11). In this way, the barrier 16 can provide additional structural support to the free ends of the leaflets, which are more centrally located, that may require more support.

FIG. 8 shows an example of an atrioventricular valve repair device 40 in a first configuration, and in a second configuration (e.g., different from the first configuration). The device 40 can be implemented in a similar manner as other devices described herein (and vice versa). As shown in FIG. 8 and similarly to the device 10 of FIG. 2, the device 40 can also have a discontinuous periphery. For example, the device 40 can include a frame 42, which has a first section 44, and second section 46 that is separated from the first section 44 (e.g., the sections 44, 46 being decoupled from each other). The device 40 also can include a barrier 48, which can include one or more filaments 50. A first end of the barrier 48 can be coupled to the section 44, while an opposing end of the barrier 48 can be coupled to the section 46. In particular, a first end of the each filament 50 can be coupled to the section 44, while an opposing second end of each filament 50 can be coupled to the section 46. As shown in the first configuration of the device 40 of FIG. 8 (e.g., the left side image), a distance 52 between the sections 44, 46 can include the barrier 48 in a non-loaded state. For example, in the first configuration of the device 40 each filament 50 can be unloaded.

In some embodiments, the device 40 can be moved (e.g., loaded) from the first configuration to the second configuration (e.g., the right side image). In the second configuration of the device 40, a distance 54 between the sections 44, 46 can be larger than the distance 52. In this way, with the increased distance 54, the barrier 48 can be tensilely loaded, which can further reduce movement of the one or more leaflets of the atrioventricular valve (e.g., the tensilely loaded barrier 48 further resisting movement of the one or more leaflets). For example, as the sections 44, 46 are moved away from each other (e.g., until reaching the second configuration), each of the filaments 50 stretch and thus are tensilely loaded and are more resisted from movement into or out of the coaptation plane.

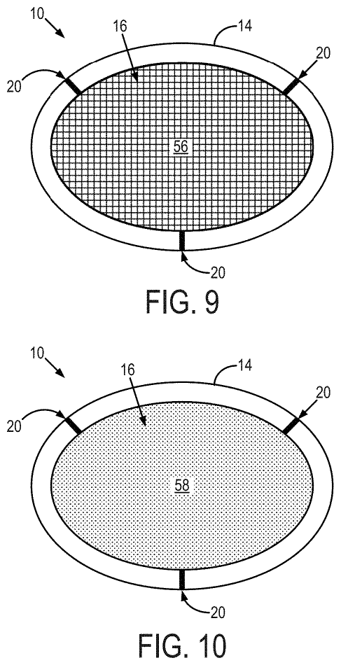

Referring to FIG. 9, in another example configuration, the atrioventricular valve repair ring 10 can include a core 12 that can define a continuous periphery. In this example, the barrier 16 is constructed from a mesh screen 56 that spans the inner volume of the atrioventricular valve repair ring 10. The mesh screen 56 can be composed of biocompatible polymer materials, such as PTFE.

The mesh screen 56 can have symmetrically distributed holes, such as holes arranged in a regularly spaced grid. In other embodiments, the mesh screen 56 can have asymmetrically distributed holes, such that there is a higher density of holes (and therefore, a higher density of screen material) in areas where it is desirable to increase the strength of the barrier 16. In some instance, the mesh screen can have a patient-specific design that accounts for the patient's specific anatomy.

Referring to FIG. 10, in still another example configuration, the atrioventricular valve repair ring 10 can include a core 12 that can define a continuous periphery. In this example, the barrier 16 is constructed as a porous sheet 58 that spans the inner volume of the atrioventricular valve repair ring 10. The porous sheet 58 can be constructed as a biocompatible membrane whose pores permit blood to flow through the porous sheet 58 while containing atrioventricular valve leaflet segments within the appropriate coaptation plane, thereby reducing (or eliminating) atrioventricular regurgitation.

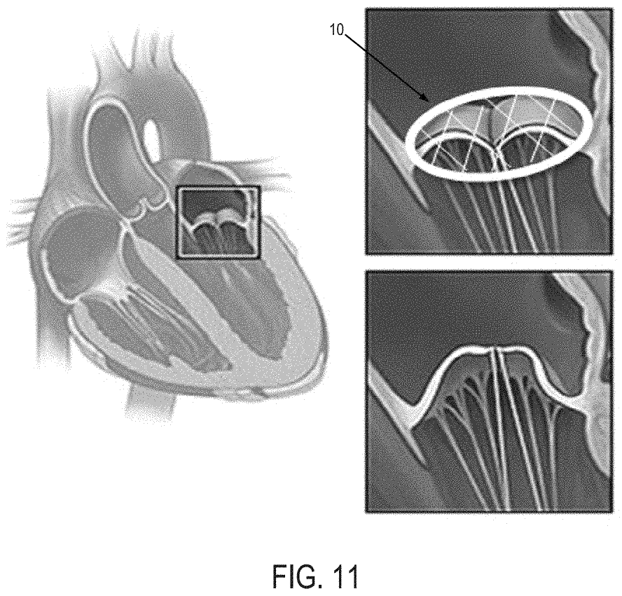

An example of a atrioventricular valve repair ring 10 that has been deployed into a patient's heart is shown in FIG. 11. The atrioventricular valve repair ring 10 is deployed around the outer periphery of the atrioventricular valve (e.g., the annulus of the atrioventricular valve) on the inflow side of the valve (i.e., within the left atrium for the mitral valve or the right atrium for the tricuspid valve). As described above, the barrier 16 of the atrioventricular valve repair ring 10 mitigates (e.g., prevents) the leaflets of the atrioventricular valve from prolapsing into the respective atrium, which has the advantage of reducing (or eliminating) atrioventricular regurgitation without requiring surgical repair, such as leaflet resection, chordal transfer/neo chords, or other complex surgical repair techniques.

Advantageously, the atrioventricular valve repair rings described in the present disclosure can be deployed via a transapical approach and/or a transseptal approaches. In an example transapical approach, a mechanism similar to the delivery of a TENDYNE™ valve (Abbott Laboratories; Abbott Park, Ill., United States) can be used to deploy the atrioventricular valve repair ring. For instance, the atrioventricular valve repair ring can be crimped on to a catheter delivery system that can be inserted into the apex of a beating heart. A pledgetted double purse string suture can be placed around the apex of the left ventricle, similar to what is performed during a transapical transcatheter aortic valve replacement (“TAVR”) procedure. A wire can then be passed across the atrioventricular annulus under fluoroscopic guidance. A sheath can be placed into the left ventricle on the beating heart. Deployment of the atrioventricular valve repair ring can be performed under transesophageal echocardiography (“TEE”) guidance with orientation of the ring to anchor in the correct orientation. An automated suturing device allows for securing the device in place.

In an example transseptal approach, a delivery system analogous to the MITRACLIP™ (Abbott Laboratories; Abbott Park, Ill., United States) system can be used. For instance, a wire can be passed across the atrial septum and into the left ventricle. Using the same type of system for securing and positioning the atrioventricular valve repair ring, the device could then be deployed entirely from the femoral or internal jugular vein.

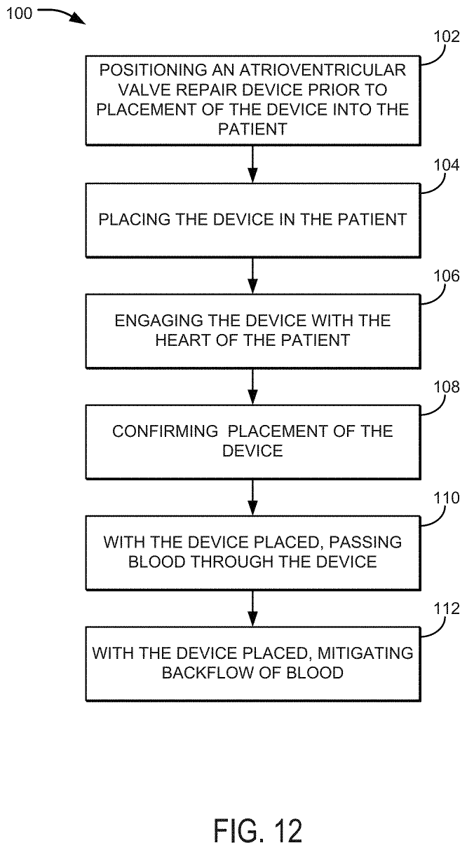

FIG. 12 shows a flowchart of a process 100 for repairing an atrioventricular valve (e.g., a tricuspid valve, a mitral valve), which can be implemented using any of the previously described an atrioventricular valve repair devices (e.g., the devices 10, 15, 40). In some configurations, the process 100 can be implemented without modifying any leaflet of the atrioventricular valve to be repaired. For example, the process 100 can be implemented without surgically dissecting, cutting, suturing, grafting, or otherwise modifying the structure of each leaflet of the atrioventricular valve to be repaired. For example, without modifying the structure of each leaflet of the atrioventricular valve can include without radially compressing the one or more leaflets of the atrioventricular valve in a direction towards the wall of the heart.

At 102, the process 100 can include positioning an atrioventricular valve repair device (e.g., the atrioventricular valve repair device 10) prior to placement of the device into the patient. For example, this can include compressing the atrioventricular valve repair device into a compact structure. In some cases, this can include placing the atrioventricular valve repair device onto a catheter system, which can include (radially) compressing the atrioventricular valve repair device so that a portion of or the entire frame of the atrioventricular valve repair device moves towards the central axis of the atrioventricular valve repair device (e.g., the frame peripherally or circumferentially being compressed towards the central axis), inserting a guidewire through the atrioventricular valve repair device (e.g., so that the frame is coaxially placed around the guidewire), and placing a sheath around the atrioventricular valve repair device (e.g., while the atrioventricular valve repair device is compressed) so that the sheath is coaxially placed around the atrioventricular valve repair device.

At 104, the process 100 can include placing the atrioventricular valve repair device into the patient. For example, this can include deploying the atrioventricular valve repair device using a trans-apical approach, a trans-atrial approach, etc. As a more specific example, in the trans-apical approach, the atrioventricular valve repair device (e.g., including portions of the catheter system including the guidewire) can be (percutaneously) inserted into an artery of a patient (e.g., a femoral artery of the patient, using, for example, a trans-femoral approach) and advanced along the artery until reaching the heart. In some cases, this can include creating a trans-apical puncture (or in other words an incision) that extends through the ventricle wall of the heart. Then, the process 100 can include advancing the atrioventricular valve repair device through the trans-apical puncture into the ventricle, through the atrioventricular valve (e.g., to be repaired), and into the atrium (e.g., that corresponds to the ventricle having the trans-apical puncture). In some cases, including when the atrioventricular valve repair device is deployed using a catheter system (or guidewire system), the guidewire can be inserted through the trans-apical puncture into the ventricle, through the atrioventricular valve, and into the atrium prior to insertion of the atrioventricular valve repair device. In this way, when advancing the atrioventricular valve repair device, the atrioventricular valve repair device can be guided along the guidewire.

As another more specific example, in the trans-atrial approach, the atrioventricular valve repair device (e.g., including portions of the catheter system including the guidewire) can be (percutaneously) inserted into an artery of the patient (e.g., a femoral artery of the patient, using, for example, the trans-femoral approach) and advanced along the artery until reaching the heart. In some cases, this can include creating a trans-atrial puncture (or in other words an incision) that extends through the atrium wall of the heart. Then, the process 100 can include advancing the atrioventricular valve repair device through the trans-atrial puncture and into the atrium. In some cases, including when the atrioventricular valve repair device is deployed using a catheter system (or guidewire system), the guidewire can be inserted through the trans-atrial puncture into the atrium, prior to insertion of the atrioventricular valve repair device. In this way, when advancing the atrioventricular valve repair device, the atrioventricular valve repair device can be guided along the guidewire.

In some embodiments, regardless of the configuration, the process 100 can include placing the atrioventricular valve repair device into an atrium of the patient proximal to the atrioventricular valve (e.g., to be repaired). For example, this can include positioning the atrioventricular valve repair device within the ventricle so that the atrioventricular valve repair device is positioned on a first side of the atrioventricular valve that is farther away from the ventricle.

At 106, the process 100 can include engaging the atrioventricular valve repair device with the heart of the patient (e.g., within the heart of the patient). This can include expanding the atrioventricular valve repair device until the atrioventricular valve repair device contacts a wall of the heart of the patient. For example, this can include expanding the frame of the atrioventricular valve repair device (e.g., from the atrioventricular valve repair device being in a compact state) until a portion (or the entire) frame contacts a wall of the heart (e.g., the atrium wall of the heart). In other cases, this can include engaging a portion of the frame of the atrioventricular valve repair device with the heart wall (e.g., when the frame of the atrioventricular valve repair device has a smaller cross-section than the cross-section of the atrium).

In some cases, the block 106 of the process 100 can include coupling the frame of the atrioventricular valve repair device to a heart wall (e.g., the atrium wall) of the patient. For example, this can include placing one or more sutures around the frame the atrioventricular valve repair device and in engagement with a wall of the heart (e.g., the ventricle wall). In some cases, this can include reinforcing the engagement between the frame of the atrioventricular valve repair device and the atrium wall of the patient (e.g., by placing sutures).

In some configurations, including when the atrioventricular valve repair device is implemented as the atrioventricular valve repair device 40, the process 100 can include coupling a first section of the frame of the atrioventricular valve repair device (e.g., the section 44) to a first side of the atrium (or ventricle) wall (e.g., by placing one or more sutures). Then, with the first section placed, this can include tensilely loading a barrier of the atrioventricular valve repair device (e.g., one or more filaments) by moving the second section of the frame away from the first section of the frame. After, this can include, with the barrier tensilely loaded, coupling a second section of the frame to an opposite side of the atrium (or ventricle) wall (e.g., by placing one or more sutures). In this way, with the barrier tensilely loaded, the one or more leaflets of the atrioventricular valve are further mitigated from movement back towards the atrium (e.g., away from the ventricle).

At 108, the process 100 can include confirming placement of the atrioventricular valve repair device. For example, including when the atrioventricular valve repair device has one or more markers, the process 100 can include acquiring one or more images of the atrioventricular valve repair device secured within the patient's heart (e.g., using an imaging system that corresponds to the markers). For example, this can include acquiring one or more fluoroscopic images of the atrioventricular valve repair device having one or more radiopaque markers to confirm placement of the atrioventricular valve repair device.

At 110, the process 100 can include (with the device placed) passing blood through the atrioventricular valve repair device. For example, this can include, passing blood from the atrium of the heart, through the atrioventricular valve repair device, through the atrioventricular valve (e.g., that has been repaired), and into the ventricle of the patient (e.g., corresponding to the atrium). In some cases, this can include passing blood (from the atrium, including when the atrium contracts), through the barrier of the atrioventricular valve repair device (e.g., through one or more holes of the barrier), through the atrioventricular valve, and into the ventricle.

At 112, the process 100 can include (with the device placed) mitigating backflow of blood from the ventricle and into the atrium through the atrioventricular valve using the atrioventricular valve repair device (e.g., during contraction of the ventricle). For example, this can include blocking movement of one or more leaflets of the atrioventricular valve with the atrioventricular valve repair device, thereby mitigating the backflow of blood. As a more specific example, this can include (e.g., when the ventricle contracts), one or more leaflets (e.g., two, three, etc.) of the atrioventricular valve contacting the barrier of the atrioventricular valve repair device and thereby blocking the one or more leaflets from further advancement into the atrium.

The present disclosure has described one or more preferred embodiments, and it should be appreciated that many equivalents, alternatives, variations, and modifications, aside from those expressly stated, are possible and within the scope of the invention.

It is to be understood that the disclosure is not limited in its application to the details of construction and the arrangement of components set forth in the following description or illustrated in the following drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

As used herein, unless otherwise limited or defined, discussion of particular directions is provided by example only, with regard to particular embodiments or relevant illustrations. For example, discussion of “top,” “front,” or “back” features is generally intended as a description only of the orientation of such features relative to a reference frame of a particular example or illustration. Correspondingly, for example, a “top” feature may sometimes be disposed below a “bottom” feature (and so on), in some arrangements or embodiments. Further, references to particular rotational or other movements (e.g., counterclockwise rotation) is generally intended as a description only of movement relative a reference frame of a particular example of illustration.

Certain operations of methods according to the disclosure, or of systems executing those methods, may be represented schematically in the FIGS. or otherwise discussed herein. Unless otherwise specified or limited, representation in the FIGS. of particular operations in particular spatial order may not necessarily require those operations to be executed in a particular sequence corresponding to the particular spatial order. Correspondingly, certain operations represented in the FIGS., or otherwise disclosed herein, can be executed in different orders than are expressly illustrated or described, as appropriate for particular embodiments of the disclosure. Further, in some embodiments, certain operations can be executed in parallel, including by dedicated parallel processing devices, or separate computing devices configured to interoperate as part of a large system.

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

As used herein, unless otherwise defined or limited, ordinal numbers are used herein for convenience of reference based generally on the order in which particular components are presented for the relevant part of the disclosure. In this regard, for example, designations such as “first,” “second,” etc., generally indicate only the order in which the relevant component is introduced for discussion and generally do not indicate or require a particular spatial arrangement, functional or structural primacy or order.

As used herein, unless otherwise defined or limited, directional terms are used for convenience of reference for discussion of particular figures or examples. For example, references to downward (or other) directions or top (or other) positions may be used to discuss aspects of a particular example or figure, but do not necessarily require similar orientation or geometry in all installations or configurations.

This discussion is presented to enable a person skilled in the art to make and use embodiments of the disclosure. Various modifications to the illustrated examples will be readily apparent to those skilled in the art, and the generic principles herein can be applied to other examples and applications without departing from the principles disclosed herein. Thus, embodiments of the disclosure are not intended to be limited to embodiments shown, but are to be accorded the widest scope consistent with the principles and features disclosed herein and the claims below. The following detailed description is to be read with reference to the figures, in which like elements in different figures have like reference numerals. The figures, which are not necessarily to scale, depict selected examples and are not intended to limit the scope of the disclosure. Skilled artisans will recognize the examples provided herein have many useful alternatives and fall within the scope of the disclosure.

The present disclosure has described one or more preferred embodiments, and it should be appreciated that many equivalents, alternatives, variations, and modifications, aside from those expressly stated, are possible and within the scope of the invention.

Various features and advantages of the disclosure are set forth in the following claims.

1. An atrioventricular valve repair ring, comprising:
a ring comprising:
a core having an arcuate shape that circumscribes an inner volume of the ring;
a sheath surrounding at least a portion of the core; and
a barrier coupled to the sheath and spanning the inner volume of the ring, wherein the barrier is constructed to permit blood to flow therethrough while preventing atrioventricular valve leaflets from passing beyond the barrier.

2. The atrioventricular valve repair ring of claim 1, wherein the core has an annular shape and defines a continuous periphery of the ring.

3. The atrioventricular valve repair ring of claim 2, wherein the core has an elliptical annular shape.

4. The atrioventricular valve repair ring of claim 1, wherein the core defines a discontinuous periphery of the ring.

5. The atrioventricular valve repair ring of claim 1, wherein the core is composed of a shape-memory material.

6. The atrioventricular valve repair ring of claim 5, wherein the core is composed of a shape-memory alloy.

7. The atrioventricular valve repair ring of claim 6, wherein the core is composed of nitinol.

8. The atrioventricular valve repair ring of claim 5, wherein the core is composed of a shape-memory polymer.

9. The atrioventricular valve repair ring of claim 1, wherein the sheath is composed of a textile material.

10. The atrioventricular valve repair ring of claim 9, wherein the textile material is a polymer-based textile material.

11. The atrioventricular valve repair ring of claim 10, wherein the polymer-based textile material comprises expanded polytetrafluoroethylene (PTFE).

12. The atrioventricular valve repair ring of claim 1, wherein the sheath fully encloses the core.

13. The atrioventricular valve repair ring of claim 1, wherein the barrier comprises a plurality of filaments.

14. The atrioventricular valve repair ring of claim 13, wherein the plurality of filaments are interwoven to form a mesh.

15. The atrioventricular valve repair ring of claim 14, wherein at least some of the plurality of filaments are looped around others of the plurality of filaments when forming the mesh.

16. The atrioventricular valve repair ring of claim 14, wherein the plurality of filaments are interwoven using a symmetrical weave pattern.

17. The atrioventricular valve repair ring of claim 14, wherein the plurality of filaments are interwoven using an asymmetrical weave pattern.

18. The atrioventricular valve repair ring of claim 13, wherein the plurality of filaments are asymmetrically distributed within the inner volume of the ring such that some portions of the barrier have a higher density of filaments than other portions of the barrier.

19. The atrioventricular valve repair ring of claim 13, wherein the plurality of filaments are coupled to the sheath by stitching the plurality of filaments to the sheath.

20. The atrioventricular valve repair ring of claim 13, wherein the plurality of filaments are composed of expanded polytetrafluoroethylene (PTFE).

21. The atrioventricular valve repair ring of claim 1, wherein the barrier comprises a mesh screen.

22. The atrioventricular valve repair ring of claim 21, wherein the mesh screen comprises a regularly spaced grid of holes in a screen material.

23. The atrioventricular valve repair ring of claim 22, wherein the screen material is composed of a biocompatible polymer.

24. The atrioventricular valve repair ring of claim 21, wherein the mesh screen comprises asymmetrically distributed holes in a screen material, such that some portions of the mesh screen have a higher density of holes than other portions of the mesh screen.

25. The atrioventricular valve repair ring of claim 24, wherein the asymmetrically distributed holes are distributed in a pattern that accounts for patient-specific anatomy.

26. The atrioventricular valve repair ring of claim 1, wherein the barrier comprises a porous sheet.

27. The atrioventricular valve repair ring of claim 1, further comprising at least one marker coupled to the ring in order to facilitate placement of the ring during a surgical procedure.

28. The atrioventricular valve repair ring of claim 27, wherein the at least one marker is composed of a radiopaque material.

29. The atrioventricular valve repair ring of claim 27, wherein the at least one marker comprises a plurality of markers that are distributed about an outer surface of the sheath.

30. An atrioventricular valve repair device comprising:
a frame;
a barrier coupled to the frame, the frame surrounding at least a portion of the barrier, the barrier having one or more holes each of which is configured to receive blood therethrough;
the device being configured to allow passage of blood from an atrium through the one or more holes of the barrier, through an atrioventricular valve, and into a ventricle during contraction of the atrium; and
wherein the barrier is configured to block advancement of one or more leaflets of the atrioventricular valve from extending farther into the atrium away from the ventricle during contraction of the ventricle.

31. The atrioventricular valve repair device of claim 30, wherein the frame has a first end and a second end opposite the first end,
wherein the barrier has a first end and an opposite second end, and
wherein the first end of the barrier is coupled to the first end of the frame, and
wherein the second end of the barrier is coupled to the second end of the frame.

32. The atrioventricular valve repair device of claim 30, wherein the frame is configured to engage at least a portion of the atrium wall of the atrium.

33. The atrioventricular valve repair device of claim 30, wherein a portion of the frame is configured to contour a curvature of the atrium wall of the atrium.

34. The atrioventricular valve repair device of claim 30, wherein the frame comprises:
a core; and
a sheath at least partially surrounding the core, the sheath being formed from a different material than the core.

35. The atrioventricular valve repair device of claim 34, wherein the barrier is coupled to at least one of the core, or the sheath.

36. The atrioventricular valve repair device of claim 30, wherein the barrier comprises a plurality of filaments with each filament overlapping an adjacent filament according to a pattern, and
wherein adjacent filaments collectively define a hole of the barrier.

37. The atrioventricular valve repair device of claim 36, each filament has a higher tensile strength than a tensile strength of the frame.

38. The atrioventricular valve repair device of claim 37, wherein each filament is tensilely loaded, and
wherein the tensile loading of each filament is greater than a tensile loading of the frame.

39. The atrioventricular valve repair device of claim 38, wherein the frame is not tensilely loaded.

40. The atrioventricular valve repair device of claim 38, wherein each filament has a cross-section that is smaller than a cross-section of the frame.

41. The atrioventricular valve repair device of claim 36, wherein each filament has a uniform cross-section along its length, and
wherein the cross-section of the frame is uniform along the entire peripheral extent of the frame.

42. The atrioventricular valve repair device of claim 36, wherein one end of each filament is coupled to one end of the frame, and an opposing end of each filament is coupled to an opposing end of the frame.

43. The atrioventricular valve repair device of claim 30, wherein the frame is configured to be compressed from a first configuration to a second configuration, and
wherein the frame is configured to expand from the second configuration to the first configuration to engage an atrium wall of the atrium.

44. The atrioventricular valve repair device of claim 30, wherein the atrioventricular valve repair device is configured to be positioned within the atrium on a first side of the atrioventricular valve that is farther away from the ventricle than a second side of the atrioventricular valve opposite the first side of the atrioventricular valve, and
wherein the atrioventricular valve repair device is configured to be coupled to a wall of the heart.
